(19) United States
(12) Patent Application Publication
(10) Pub. No.: US 2026/0000708 A1
(43) Pub. Date: Jan. 1, 2026

(54) Copper Hydrogel Beads as a Diet Supplement

(71) Applicant: Texas Tech University System, Lubbock, TX (US)

(72) Inventors: Maria A. Calle, Amarillo, TX (US); Jon Thompson, Amarillo, TX (US); Maria Fernandez de la Garza, Lubbock, TX (US)

(21) Appl. No.: 18/707,899

(22) PCT Filed: Nov. 8, 2022
(86) PCT No.: PCT/US2022/049203

§ 371 Date: May 7, 2024

Related U.S. Application Data
(60) Provisional application No. 63/276,836, filed on Nov. 8, 2021.

(51) Int. Cl. A61K 33/34; A23K 20/147; A23K 20/163; A23K 20/195; A23K 20/20; A23K 50/30; A61K 9/06; A61K 47/36; A61K 47/42; A61P 31/04

(52) U.S. Cl. CPC A61K 33/34; A23K 20/147; A23K 20/163; A23K 20/195; A23K 20/30; A23K 50/30; A61K 9/06; A61K 47/36; A61K 47/42; A61P 31/04

(57) ABSTRACT

The present invention includes a feed, a feed additive, and method of reducing or eliminating pathogenic bacteria in a mammal comprising providing the mammal with a composition comprising a hydrogel and an amount of copper effective to, e.g., reduce or eliminate pathogen shedding in a mammal, wherein the copper comprises between 0.01 to 100 mg/g of the hydrogel.

### CROSS-REFERENCE TO RELATED APPLICATIONS

This application claims priority to U.S. Provisional Application Ser. No. 63/276,836, filed Nov. 9, 2021, the entire contents of which are incorporated herein by reference.

### TECHNICAL FIELD OF THE INVENTION

The present invention relates in general to the field of diet supplementation to reduce pathogen shedding, improve nutrition, and more particularly, to the use of copper beads as a diet supplement to reduce pathogen presence and shedding in mammals.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with nutrition and pathogenic bacteria in animals.

United States Patent Application Publication No. 20040115177, filed by Harris, et al., entitled “Probiotic compositions and methods against bacterial infection in livestock animals”, is directed to methods of administering probiotic bacteria to livestock animals in an amount effective to reduce the amount of hazardous bacteria found in gut-associated lymph nodes. Probiotic bacteria in an acceptable carrier are administered to the livestock at a selected time before transportation of the animal for harvest or other purposes. The invention also provides compositions comprising probiotic bacteria in an amount effective to reduce the amount of Salmonella in livestock animals, e.g., in their feces.

United States Patent Application Publication No. 20100047209, filed by Stanton, et al., entitled “Probiotic composition suitable for animals”, is directed to a probiotic composition to alleviate Salmonella infection in farm animals. The composition may comprise at least one of Lactobacillus murinus. Lactobacillus pentosus. Lactobacillus salivarius sub-species salivarius, and Pediococcus pentosaceus. The composition may be formulated as an animal feedstuff, or as a pharmaceutical composition.

United States Patent Application Publication No. 20110256116, filed by Ware, et al., entitled “Low/High Dose Probiotic Supplements And Methods Of Their Use”, is directed to methods and compositions are hereby disclosed for reducing the numbers of E. coli O157:H7, Salmonella or other pathogens in an animal. The methods include administering to the animal a lactic acid producing bacterium at a relatively low dosage in combination with a lactate utilizing bacterium, followed by administration of the lactic acid producing bacterium at a relatively high dosage. The methods disclosed help achieve pre-harvest food safety and enhance feed performance while keeping the total cost relatively low. The preferred lactic acid producing bacterium is Lactobacillus acidophilus animalis and the preferred lactate utilizing bacterium is Propionibacterium freudenreichii.

U.S. Pat. No. 7,063,836, issued to Garner, et al., entitled, “Compositions and methods for inhibiting pathogenic growth,” is directed to methods and compositions for treating an animal to inhibit the incidence and growth of E. coli O157:H7 and other pathogenic bacteria. The method of treatment includes administering a therapeutically effective amount of Lactobacillus acidophilus or one or a combination of a number of other probiotic bacteria to an animal. An alternative treatment method comprises administering a therapeutically effective amount of a lactic acid producing bacterium such as Lactobacillus acidophilus in combination with a lactate utilizing bacterium such as Propionibacterium freudenreichii.

### SUMMARY OF THE INVENTION

As embodied and broadly described herein, an aspect of the present disclosure relates to a feed additive comprising a hydrogel and an amount of copper effective to reduce or eliminate pathogen shedding in a mammal, wherein the copper comprises between 0.01 to 100 mg/g of the hydrogel. In one aspect, the mammal is bovine, equine, ovine, porcine, or caprine. In another aspect, the hydrogel comprises at least one of: alginate, pectin, gelatin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof. In another aspect, the feed additive further comprises adding the feed additive to a base feed comprises at least one element selected from the group consisting of corn, sorghum, barley, wheat, soybean, peanut, canola, fish meal, milk products, fats, oils, vitamins, minerals and mixtures thereof. In another aspect, the feed additive further comprises at least one feed component selected from: carbohydrates, fats, minerals, proteins, roughage, silage, vitamins, and trace elements. In another aspect, the copper releases at least 60, 70, 80, 90, 100, 110, 120, 130, 140, 150% more copper than unmodified alginate beads over 1, 2, 3, 4, 5, or 6 hours when dissolved in simulated gastric fluid. In another aspect, at least 10, 20, 30, 40, 50, 60, 70, 75, 80, 90, or 100% of the copper is released over 1, 2, 3, 4, 5, 6, 8 or 12 hours in simulated gastric fluid. In another aspect, the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g. In another aspect, the pathogenic bacteria are selected from at least one of Salmonella sp., E. coli O157:H7, generic E. coli, or Enterobacteriaceae. In another aspect, the hydrogel is a bead that does not comprise calcium. In one aspect, the copper is loaded into the hydrogel while in solution and after the copper is loaded into the hydrogel, the hydrogel is dried.

As embodied and broadly described herein, another aspect of the present disclosure relates to a method of eliminating or reducing pathogenic bacteria in a mammal comprising: identifying an animal in need of eliminating or reducing pathogenic bacteria in a gut of the animal; and providing the animal with an effective amount of feed additive comprising a hydrogel and an amount of copper effective to reduce or eliminate pathogen shedding in a mammal sufficient to reduce or eliminate the pathogenic bacteria in the gut. In one aspect, the copper comprises between 0.01 to 100 mg/g of the hydrogel. In another aspect, the mammal is bovine, equine, ovine, porcine, or caprine. In another aspect, the hydrogel comprises an alginate, gelatin, pectin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof. In another aspect, the method further comprises adding the feed additive to a base feed comprises at least one element selected from the group consisting of corn, sorghum, barley, wheat, soybean, peanut, canola, fish meal, milk products, fats, oils, vitamins, minerals and mixtures thereof. In another aspect, the method further comprises adding at least one feed component selected from: carbohydrates, fats, minerals, proteins, roughage, silage, vitamins, and trace elements. In another aspect, the copper releases at least 60, 70, 80, 90, 100, 110, 120, 130, 140, 150% more copper than unmodified alginate beads over 1, 2, 3, 4, 5, or 6 hours when dissolved in simulated gastric fluid. In another aspect, at least 10, 20, 30, 40, 50, 60, 70, 75, 80, 90, or 100% of the copper is released over 1, 2, 3, 4, 5, 6, 8 or 12 hours in simulated gastric fluid. In another aspect, the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g. In another aspect, the pathogenic bacteria are selected from at least one of Salmonella sp., E. coli O157:H7, generic E. coli, or Enterobacteriaceae. In another aspect, the hydrogel is a bead that does not comprise calcium. In one aspect, the copper is loaded into the hydrogel while in solution and after the copper is loaded into the hydrogel, the hydrogel is dried.

As embodied and broadly described herein, another aspect of the present disclosure relates to a feed comprising a hydrogel and an amount of copper effective to reduce or eliminate pathogen shedding in a mammal, wherein the copper comprises between 0.01 to 100 mg/g of the hydrogel and at least one feed component selected from: carbohydrates, fats, minerals, proteins, roughage, silage, vitamins, and trace elements. In one aspect, the mammal is bovine, equine, ovine, porcine, or caprine. In another aspect, the hydrogel comprises at least one of: alginate, pectin, gelatin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof. In another aspect, the feed further comprises a base feed comprises at least one element selected from the group consisting of corn, sorghum, barley, wheat, soybean, peanut, canola, fish meal, milk products, fats, oils, vitamins, minerals and mixtures thereof. In one aspect, the copper is loaded into the hydrogel while in solution and after the copper is loaded into the hydrogel, the hydrogel is dried. In another aspect, the copper is 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g of the hydrogel when wet or after it is dried.

As embodied and broadly described herein, another aspect of the present disclosure relates to a method of making a feed comprising loading into a hydrogel an amount of copper effective to reduce or eliminate pathogen shedding in a mammal, wherein the copper comprises between 0.01 to 100 mg/g of the feed. In one aspect, the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g. In one aspect, the copper is loaded into the feed while in solution and after the copper is loaded into the feed, the feed is dried.

### BRIEF DESCRIPTION OF THE DRAWINGS

For a more complete understanding of the features and advantages of the present invention, reference is now made to the detailed description of the invention along with the accompanying figures and in which:

FIG. 1A is a photo showing alginate beads formulated with copper. FIG. 1B is a photo showing alginate beads formulated with copper after drying (time-release formulation).

FIG. 2 is a graph that shows the copper release measured on three formulations of beads in simulated gastric fluid (SGF). Hydration state of beads affected time-release of copper, with dehydrated beads more slowly releasing copper in SGF.

FIG. 3 is a graph showing time-release formulation compared to copper sulfate alone against Salmonella cells in vitro.

FIG. 4 is a graph showing in vitro efficacy of copper alginate beads on a co-culture of Lactic Acid Bacteria (LAB) and Salmonella.

FIG. 5 is a graph that shows the copper present in swine feces analyzed by atomic absorption spectroscopy after collection.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the terms “subject,” “patient” and “individual” are used interchangeably herein, to refer to a mammal (e.g., livestock, such as, cattle, pigs, sheep, goats, bison, rabbit, turkey, goose, duck, or chicken) or fish to be treated or feed the additive of the present invention, which may be provided in wet or dry form.

As used herein, the terms “effective amount” and “safe and effective amount” refer to the quantity of the present invention that is sufficient to yield a desired response without undue adverse side effects within a reasonable benefit/risk ratio. Non-limiting examples of adverse effects include viral, bacterial or fungal infections, weight-loss, or intestinal disorders.

As used herein, the term “treatment” refers to the application or administration of the copper loaded into a hydrogel of the present invention to a subject, or application or administration of the therapeutic agent to a subject that may develop a disease or infection or has a disease, symptom or predisposition to a disease. The treatment is provided to the subject to prevent, cure, heal, alleviate, relieve, alter, remedy, ameliorate, improve or affect the disease, the symptoms of disease, or the predisposition toward disease. For example, “treatment” of a subject with no symptoms or clinically manifestations of a disease or disorder have been identified is preventive or prophylactic therapy. In one example, the copper loaded into a hydrogel of the present invention is sufficient to reduce or eliminate the microbial contamination in the gut.

As used herein, the terms “additive” and “feed additive” are used to describe compositions that may be used in conjunction with animal feed as feed additive resulting in an improvement of the health of livestock, poultry and fish, and a reduction or elimination of microbial contamination in non-gut associated lymph nodes. The additive or feed additive for the domestic animals, poultry and fish may be of powder, grain or liquid form and will be used in accordance with the feeding condition and installations of the farm and the target animal.

Suitable animal feedstuffs for adding the present invention include, e.g., green feed, silages, dried green feed, roots, tubers, fleshy fruits, grains and seeds, brewer's grains, pomace, brewer's yeast, distiller's spent grains, milling byproducts, byproducts of the production of sugar, starch and oil recovery and various food wastes. The feed additive of the present invention may be used alone or in conjunction with other well-known feed additives such as antioxidants or mixtures of various substances (mineral mixtures, vitamin mixtures) that can be added to such feeds for enhancement. Specific feeds may also be adapted for certain animal species depending on age and stages of development.

Base feeds suitable for use in conjunction with copper loaded into a hydrogel of the present invention may be prepared as is well-known to the artisan skilled in the art of preparing feeds, e.g., they may use those as described in Kirk-Othmer, Encyclopedia of Chemical Technology, 4th Ed., vol. 10, pp. 288-300, Wiley, N.Y., 1993, relevant portions incorporated herein by reference. For example, the base feed may include one or more of the following ingredients: corn, sorghum, barley, wheat, soybean, peanut, canola, fish meal, milk products, fats and oils, vitamins and minerals.

In one aspect, the copper is loaded into the hydrogel while in solution and after the copper is loaded into the hydrogel, the hydrogel is dried. In another aspect, the copper is 0.1, 0.2, 0.3, 0.4, 0.5, 0.75, 0.8, 0.9, 1, 2, 3, 4, 5, 6, 7, 8, 9, 10, 11, 12, 13, 14, 15, 20, 25, 30, 35, 40, 45, 50, 60, 65, 70, 75, 80, 95, 90, 95 or 100 mg/g of the hydrogel when wet or after it is dried.

### Example 1. Use of Copper Hydrogel Beads as a Diet Supplement to Reduce Pathogen Shedding in Pigs

The present inventors have developed an economically viable strategy for pre-harvest interventions including changes in production practices and novel feed additives. The present inventors investigated the dose of copper beads required to reduce Salmonella sp., and E. coli O157 using in vitro experiments. Next, the inventors can test the effectiveness of supplementing copper beads, to reduce the concentration of Salmonella sp. and E. coli O157 in feces of feeder pigs.

Copper is known to be an essential nutrient in animals, participating in oxidation-reduction reactions, transport of oxygen, synthesis of hemoglobin, and protection against oxidative stress¹⁻². In pigs, copper is important for maintenance, growth, and reproduction³. It is often administered during feeding to improve growth rate and performance, especially during early growth phases⁴⁻⁷. This metal is widely known for its antimicrobial properties: thus, it has been recognized by the Environmental Protection Agency as an antimicrobial agent⁸. Further, copper is used for weight gain during a fattening period, also referred to as increasing the performance of pigs. The improved delivery of copper leading to weight gain. By way of explanation, but in no way a limitation of the present invention, the presence of copper in livestock waste pits may decrease ammonia emissions.

Using the present invention, it was found that encapsulating copper in alginate beads is an effective intervention to deliver a slow-release of copper ions into the GI tract of pigs. When copper is ingested directly it is absorbed rapidly and a minimum amount of copper will advance through the intestines, where most foodborne bacterial pathogens reside. The time-release alginate beads will deliver antibacterial copper at a rate to reach deep into the small and large intestines of the animal.

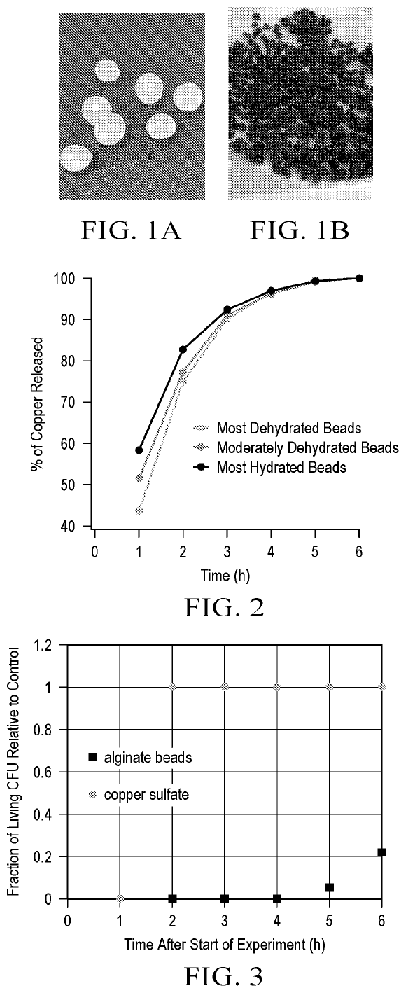

FIG. 1A is a photo showing alginate beads formulated with copper. FIG. 1B is a photo showing alginate beads formulated with copper after drying (time-release formulation).

Copper alginate beads were successfully prepared and the dose of copper within the beads is adjustable based on formulation or application rate. An initial in vitro study was conducted demonstrating >4-6 Log bacterial reduction by copper alginate beads (TABLE 2). In addition, extended time-release of copper ions from a variety of alginate beads has been demonstrated (FIG. 2).

FIG. 2 is a graph that shows the copper release measured on three formulations of beads in simulated gastric fluid (SGF). Hydration state of beads affected time-release of copper, with dehydrated beads more slowly releasing copper in SGF. A further in vitro experiment was conducted and demonstrated copper alginate beads to release sufficient copper to kill approximately 80% of Salmonella bacteria in vitro for over 6 hours after application compared to copper sulfate alone which is seen to dissolve very rapidly and is therefore only effective for a short time (FIG. 3).

Preparation of beads: Briefly, copper alginate beads can be prepared by using a drop method. One non-limiting example is an aqueous slurry containing alginate, pectin, and optionally collagen peptides, which was prepared and added to a bath solution containing various concentrations of copper ions dropwise. Hydrogel beads form immediately upon immersion in the copper-containing bath solution. The pectin and peptides are cross-linked using the method of Levy and Edwards-Levy (1996)⁹. The beads may be, e.g., a solution of alginate, pectin, and a polyamino-crosslinker, each at a weight percent selected to form beads. In this example, the beads were made from a solution of alginate (1%), pectin (2%), and collagen (3%), that was dropped into an aqueous solvent to form beads upon contact with the aqueous solvent that contained the copper in solution, in this example, copper sulfate.

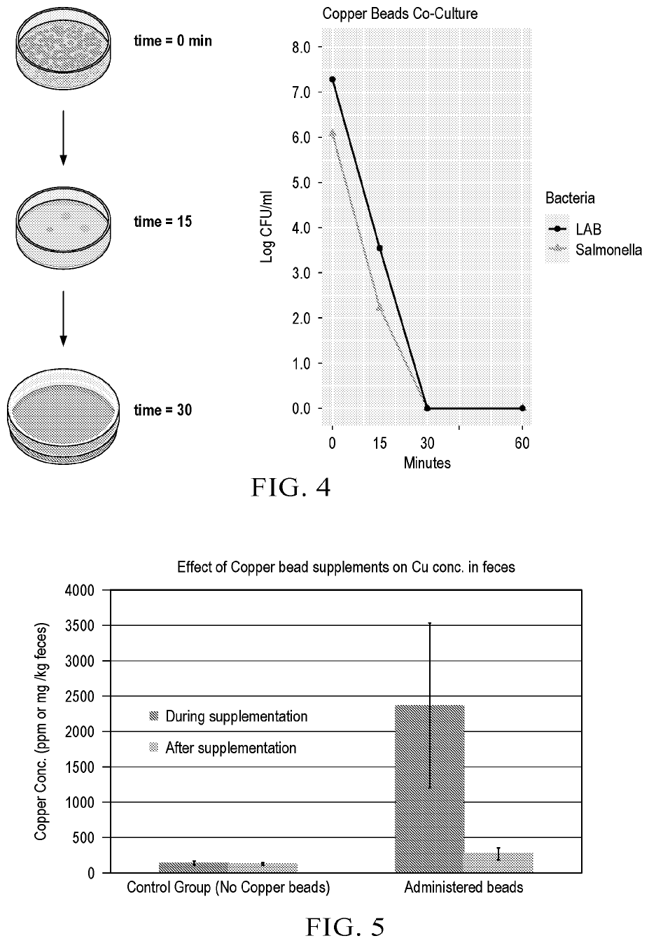

Test beads efficacy in laboratory: Different formulas of copper beads were tested to determine bacterial reduction. Bacterial cultures were prepared independently for Salmonella sp. And E. coli O157:H7, (freshy made prior to experiments). A known mass/copper concentration of beads was added into a test tube containing the bacterial culture, and different beads-bacteria ratios were tested. Data is reported as Log reduction and % of bacterial destruction. Copper alginate beads were also found to be effective in vitro against a co-culture of Lactic Acid Bacteria (LAB) and Salmonella (FIG. 4). Briefly, LAB (gram-positive) and Salmonella (gram-negative) were mixed to prepare a co-culture bacterial cocktail. To prepare this cocktail, LAB strains and Salmonella strains were grown individually in Tryptic Soy Broth (TSB) for 48 h at 37° C. Four LAB strains were combined by adding equal aliquots of each into a sterile tube. Similarly, a Salmonella cocktail was made with three different strains by combining equal volumes of each into a sterile tube. The co-culture was made by mixing a 1:1 ratio of the LAB and Salmonella cocktails into a sterile tube. A volume of 10 mL of the co-culture cocktail was combined with 2 g of copper beads. Microbial survival in the copper beads mix was tested over time. The co-culture cocktail was enumerated at time points of 0, 15, 30, and 60 min. Time 0 allows us to establish the original microbial concentration. Times 15, 30, and 60 min, will provide information pertaining killing rate in contact with the copper treatment. At each corresponding time point, LABs were enumerated on De Man, Rogosa and Sharpe agar (MRS) and Salmonella was enumerated on Xylose Lysine Tergito-4 (XLT4) agar. The initial concentration of LAB and Salmonella at was 7.28 Log CFU/ml and 6.07 Log CFU/ml, respectively (observed at time point 0 min). Within the first 15 minutes, a decrease of 3.87 and 3.73 Log CFU/ml was observed in Salmonella and LAB, respectively. This indicates that both Salmonella and LABs were inhibited by the copper bead in-vitro within 15 min. After 30 min, no viable bacteria were recovered from the culture mix, which indicates that copper beads successfully reduced both microorganisms (FIG. 4).

One of the main concerns of administering antimicrobials in animal feed is the occurrence of antimicrobial resistant bacteria. By way of explanation, and in no way a limitation of the present invention it is possible that a benefit of using copper may be that microorganisms appear to not develop resistance to this mineral. Copper is a low-cost alternative, which will benefit animal growth/performance, health, while controlling the shedding of microbial pathogens.

TABLE 1
Evaluation of different copper formulas and
concentration of copper present in the beads

| | Mass of beads used for | Indicated copper per |
|---|---|---|
| Bath Composition | analysis testing (g) | gram of beads¹ (mg) |
| ONLY calcium @ 10 g | 1.93 | <0.05 |
| CaCl₂ per 100 mL | | |
| ONLY copper (1.0M), | 1.63 | 11 |
| no calcium added | | |
| ONLY copper (0.4M), | 1.53 | 6.2 |
| no calcium added | | |

These results show that the mass of copper present in each bead type varies, however, not necessarily linearly with concentration of copper. Nonetheless, the copper content of the beads can be tuned to a specific desired level.

TABLE 2
Bacterial reduction using a model with an E. coli surrogate¹

| | Reduction after 12 | Reduction after 24 |
|---|---|---|
| | hours of exposure | hours of exposure |
| Copper bead preparation | (Log CFU/ml) | (Log CFU/ml) |
| ONLY copper (1.0M), | 6.06 | 6.86 |
| no calcium added | | |
| ONLY copper (0.4M), | 3.59 | 3.76 |
| no calcium added | | |

¹Non-pathogenic E. coli indicator panel (ATCC MP-26TM), serves as surrogate for Salmonella and E. coli O157:H7.

Copper in Feces Analysis. A field trial was conducted. Copper present in swine feces was analyzed by atomic absorption spectroscopy after collection. A time-release formulation of copper beads was added to the swine feed for consumption with a target of administering 20 g of beads per day, per animal. The target was 225 ppm in feed or 225 mg/kg feed.

This study was divided into three weeks or 21 days. 1) Baseline week, 2) Supplementation week, and 3) Return to baseline week. Fecal samples were collected from each animal at 8 pre-determined sampling points. During the first week, animals were weighed and moved into their experimental pen. Following pen assignments, fecal samples were collected on days 5 and 6. On day 8 of the study, copper beads were supplemented in feed to the treatment group, and animals were weighted at the beginning of treatment. Copper beads were fed from day 8-14 and samples were collected every other day. At the end of week 2, animals were weighed at the end of treatment, and copper beads were removed from feed. Finally, on the third week, once the copper beads were removed from the treatment group, four days were allowed to pass until samples were collected again. This was to observe if there was a return to the baseline in the samples after the copper treatment.

FIG. 5 shows the copper in ppm present in dry feces. Within the control group of animals there is no significant differences in copper content. However, during supplementation with the copper alginate beads, the fecal concentration of copper in the treatment group spiked to over 2,000 ppm. After diet supplementation ceased, the level of copper dropped significantly in the test group. This demonstrates that the copper alginate supplement was effective at raising copper concentration within the GI tract.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Espinosa, C. D., Stein, H. H. Digestibility and metabolism of copper in diets for pigs and influence of dietary copper on growth performance, intestinal health, and overall immune status: a review. J Animal Sci Biotechnol 12, 13 (2021).
2. Hill G M. Minerals and mineral utilization in swine. In: Chiba L I, editor. Sustainable swine nutrition. Oxford: Blackwell Publishing Ltd: 2013. p. 186-9.
3. Suttle N F. Mineral nutrition of livestock. 4th ed. Oxfordshire: CABI Publishing: 2010.
Jacela J Y, DeRouchey J M, Tokach M D, et al. Feed additives for swine: Fact sheets-high dietary levels of copper and zinc for young pigs, and phytase. J Swine Health Prod. 2010: 18 (2): 87-91.
4. National Research Council. 1998. In: Nutrient Requirements of Swine. 10th ed. Washington, D C: National Academy Press.
5. Cromwell, G. L., H. J. Monegue, and T. S. Stahly. 1993. Long-term effects of feeding a high copper diet to sows during gestation and lactation. J. Anim. Sci. 71:2996-3002.
6. Hill G M. Minerals and mineral utilization in swine. In: Chiba L I, editor. Sustainable swine nutrition. Oxford: Blackwell Publishing Ltd: 2013. p. 186-9.
7. Montero, D. A., Arellano, C., Pardo, M. et al. Antimicrobial properties of a novel copper-based composite coating with potential for use in healthcare facilities. Antimicrob Resist Infect Control 8, 3 (2019).
8. Levy M C, Edwards-Levy F. Coating alginate beads with cross-linked biopolymers: a novel method based on a transacylation reaction. J Microencapsul. 1996 March-April, 13 (2): 169-83.
9. FDA, 2020. Title 21 Part 184. Direct Food Substances Affirmed as Generally Recognized as Safe. Accessed on: Aug. 19, 2021. Available at: www.accessdata.fda.gov/scripts/cdrb/cfdocs/cfcfr/CFRSearch.cfm?CFRPart=184

1. A feed additive comprising a hydrogel and an amount of copper effective to reduce or eliminate pathogen shedding in a mammal, wherein the copper comprises between 0.01 to 100 mg/g of the hydrogel.

2. The feed additive of claim 1, wherein the mammal is bovine, equine, ovine, porcine, or caprine.

3. The feed additive of claim 1, wherein the hydrogel comprises at least one of: alginate, pectin, gelatin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof.

4. The feed additive of claim 1, further comprising at least one of:
a base feed that comprises at least one element selected from the group consisting of corn, sorghum, barley, wheat, soybean, peanut, canola, fish meal, milk products, fats, oils, vitamins, minerals and mixtures thereof; or
at least one feed component selected from: carbohydrates, fats, minerals, proteins, roughage, silage, vitamins, and trace elements.

5. (canceled)

6. The feed additive of claim 1, wherein at least one of:
the copper releases at least 60, 70, 80, 90, 100, 110, 120, 130, 140, 150% more copper than unmodified alginate beads over 1, 2, 3, 4, 5, or 6 hours when dissolved in simulated gastric fluid;
at least 10, 20, 30, 40, 50, 60, 70, 75, 80, 90, or 100% of the copper is released over 1, 2, 3, 4, 5, 6, 8 or 12 hours in simulated gastric fluid;
the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g; or
the copper is loaded into the hydrogel while in solution and after the copper is loaded into the hydrogel, the hydrogel is dried.

7. (canceled)

8. (canceled)

9. The feed additive of claim 1, wherein the pathogenic bacteria are selected from at least one of Salmonella sp., E. coli O157:H7, generic E. coli, or Enterobacteriaceae.

10. The feed additive of claim 1, wherein the hydrogel is a bead that does not comprise calcium.

11. (canceled)

12. A method of eliminating or reducing pathogenic bacteria in a mammal comprising: identifying an animal in need of eliminating or reducing pathogenic bacteria in a gut of the animal; and providing the animal with an effective amount of feed additive comprising a hydrogel and an amount of copper effective to reduce or eliminate pathogen shedding in a mammal sufficient to reduce or eliminate the pathogenic bacteria in the gut.

13. The method of claim 12, wherein the copper comprises between 0.01 to 100 mg/g of the hydrogel.

14. The method of claim 12, wherein the mammal is bovine, equine, ovine, porcine, or caprine.

15. The method of claim 12, wherein the hydrogel comprises an alginate, gelatin, pectin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof.

16. The method of claim 12, further comprising at least one of:
adding the feed additive to a base feed comprises at least one element selected from the group consisting of corn, sorghum, barley, wheat, soybean, peanut, canola, fish meal, milk products, fats, oils, vitamins, minerals and mixtures thereof; or
adding at least one feed component selected from: carbohydrates, fats, minerals, proteins, roughage, silage, vitamins, and trace elements.

17. (canceled)

18. The method of claim 12, wherein at least one of:
the copper releases at least 60, 70, 80, 90, 100, 110, 120, 130, 140, 150% more copper than unmodified alginate beads over 1, 2, 3, 4, 5, or 6 hours when dissolved in simulated gastric fluid;
at least 10, 20, 30, 40, 50, 60, 70, 75, 80, 90, or 100% of the copper is released over 1, 2, 3, 4, 5, 6, 8 or 12 hours in simulated gastric fluid;
the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g; or
the copper is loaded into the hydrogel while in solution and after the copper is loaded into the hydrogel, the hydrogel is dried.

19. (canceled)

20. (canceled)

21. The method of claim 12, wherein the pathogenic bacteria are selected from at least one of Salmonella sp., E. coli O157:H7, generic E. coli, or Enterobacteriaceae.

22. The method of claim 12, wherein hydrogel is a bead that does not comprise calcium.

23. (canceled)

24. A feed comprising a hydrogel and an amount of copper effective to reduce or eliminate pathogen shedding in a mammal, wherein the copper comprises between 0.01 to 100 mg/g of the hydrogel and at least one feed component selected from: carbohydrates, fats, minerals, proteins, roughage, silage, vitamins, and trace elements.

25. The feed of claim 24, wherein the mammal is bovine, equine, ovine, porcine, or caprine.

26. The feed of claim 24, wherein the hydrogel comprises at least one of: alginate, pectin, gelatin, polyvinyl alcohol, polylactide, poly(ethylene glycol), poly(glycerol sebacate), chitin, collagen, fibrin, fibronectin, tropoelastin, chitosan, hyaluronic acid, agarose, starch, glucan, polyphosphazene, polyacrylic acid and derivatives thereof, polyamino acid, degradable polyurethane, polysaccharides, polyphosphazenes, poly(acrylic acids), poly(methacrylic acids), copolymers of acrylic acid and methacrylic acid, poly(alkylene oxides), poly(vinyl acetates), polyvinylpyrrolidones, copolymers and blends thereof, and combinations thereof.

27. The feed of claim 24, further comprises a base feed comprises at least one element selected from the group consisting of corn, sorghum, barley, wheat, soybean, peanut, canola, fish meal, milk products, fats, oils, vitamins, minerals and mixtures thereof.

28. A method of making a feed comprising loading into a feed and an amount of copper effective to reduce or eliminate pathogen shedding in a mammal, wherein the copper comprises between 0.01 to 100 mg/g of the feed.

29. The method of claim 28, wherein the copper is at 0.2, 0.3, 0.4, 0.5, 0.75, 0.8, 0.9, 1.0, 2.0, 3.0, 4.0, 5.0, 6.0, 7.0, 7.5, 8.0, 9.0, 10, 12.5, 15, 20, 30, 40, or 45, 50, 60, 70, 75, 80, 90, or 95 mg/g of the feed.

30. The method of claim 28, wherein the copper is loaded into the feed while in solution and after the copper is loaded into the feed, the feed is dried.
